(19) United States
(12) Patent Application Publication
(10) Pub. No.: US 2006/0083794 A1
(43) Pub. Date: Apr. 20, 2006

(54) Use of an extract from the plant argania spinosa

(76) Inventors: Florence Henry, Villers-les-Nancy (FR); Louis Danoux, Saulxures les Nancy (FR); Gilles Pauly, Nancy (FR); Zoubida Charrouf, Rabat R.P (MA)

Correspondence Address: COGNIS CORPORATION;PATENT DEPARTMENT, 300 BROOKSIDE AVENUE, AMBLER, PA, 19002, US

(21) Appl. No.: 10/539,879

(22) PCT Filed: Dec. 9, 2003
(86) PCT No.: PCT/EP03/13921

§ 371 Date: Jun. 16, 2005

(30) Foreign Application Priority Data
Dec. 18, 2002 (EP) 02293130.7

(51) Int. Cl. A61K 36/185

(52) U.S. Cl. 424/725

(57) ABSTRACT

A method and preparation which uses Argania spinosa is provided for treating acne, seborrhoea, unwanted hair, and having anti-5-α-reductase activity. The useful compositions contain from 0.01 to 25% by weight, based on a dry weight, of an extract of Argania spinosa.

### FIELD OF THE INVENTION

This invention relates generally to cosmetic and/or dermopharmaceutical care products and, more particularly, to the use of extracts of the plant Argania spinosa for the production of anti-acne and/or anti-seborrhoea preparations and preparations with anti-5-α-reductase activity.

### PRIOR ART

Greasy and acne-affected skin shows increased secretion of sebum and tallow through over-activity of the sebaceous glands. The triglycerides present in the secretions of the sebaceous glands are decomposed on the skin by lipases of various microorganisms such as, for example, Corynebacterium acnes, Staphylococcus epidermis and Pytirosporum ovale and free fatty acids are released. Some of these free fatty acids lead to the characteristic inflammatory phenomena of the acute stage of acne.

The conversion of testosterone into 5-dihydrotestosterone (5-DHT) by the enzyme 5-α-reductase has been found to be one of the causes of increased sebaceous gland secretion. Accordingly, the activity of the enzyme 5-α-reductase, which can be found in particular in the sebaceous glands and in apocrine glands and in keratinocytes and fibroblasts, is of particular importance to skin affected by acne or seborrhoea.

Today, cosmetic preparations are available to the consumer in a variety of combinations. Nevertheless, there is a need on the market for products with an improved performance spectrum. In this connection, consumers demand dermatological compatibility and the use of natural products. In addition, it is desirable to obtain distinctly better products by combining already known active principles or by discovering new applications for already known classes of substances. More particularly, extracts of plants and their ingredients are being more commonly used in cosmetic and pharmaceutical products. However, there are many plants and their potential effects which have yet to be discovered and many new applications of already known classes of substances are time and again causing surprise.

It has been known for some time that many saponins obtained from various plants and microorganisms show anti-radical, analgesic and also anti-inflammatory activity. Such activity was also demonstrated for the saponins isolated from Argania spinosa by Alaoui et al. [Alaoui K. et al.; Annales pharmaceutique francaices, 1998, 56, 220-228]. In addition, some saponins have been found to show antibiotic and fungistatic activity. Saponins and especially the triterpene saponins are made up of a tetra- or pentacyclic triterpene aglycon and one or two glycosidically linked sugar chains.

The problem addressed by the present invention was to find new applications for highly compatible extracts of renewable vegetable raw materials rich in active components, more particularly active components for the treatment of acne-affected and seborrhoeic skin.

### DESCRIPTION OF THE INVENTION

The present invention relates to the use of extracts of the plant Argania spinosa (L.) Skeels, hereinafter referred to in short as Argania spinosa, for the production of anti-acne preparations, for the production of anti-seborrhoea preparations and for the production of preparations with anti-5-α-reductase activity.

It has surprisingly been found that preparations with an excellent effect in anti-acne preparations and in anti-seborrhoea preparations, coupled with high dermatological compatibility, can be produced by using extracts of the plant Argania spinosa. Accordingly, they may be used with outstanding effect against greasy skin, acne skin and greasy scalps, even in people with sensitive skin. The preparations produced show distinct anti-5-α-reductase activity. Accordingly, a particular embodiment of the present invention is the use of extracts of the plant Argania spinosa for the production of preparations for treating unwanted hair growth in women, more particularly unwanted hair growth occurring after the menopause. This unwanted hair growth occurs in particular on the face, above all around the mouth, or on the legs. Unwanted excessive hair growth in women, particularly after the menopause, is associated with hyperactivity of 5-α-reductase. The unwanted, often excessive hair growth on certain parts of the face or on the legs can thus be effectively inhibited by using extracts of the plant Argania spinosa.

Argania spinosa

The extracts used in accordance with the invention are obtained from plants of the family Sapotaceae, more especially from Argania spinosa (L.) Skeels, hereinafter referred to simply as Argania spinosa. This plant is a tree which is mainly found in Morocco on the western side of the Atlas mountains. On its gnarled trunks and thorny branches, it forms berries the size and shape of olives with one or two seed kernels. The nutty-tasting oil from the seed kernels is used inter alia as an edible oil.

In the context of the present invention, the term plant is intended to encompass both whole plants and plant parts (leaves, roots, stem, bark, flowers, fruits, fruit flesh and seed kernels) and mixtures thereof. According to the invention, the seed kernels of the fruit of the plant, more particularly the extraction of the residue from defatted seed kernels, are particularly preferred for the extraction of the saponins.

Saponins

Saponins in the context of the present invention are, basically, any saponins which can be isolated from the plant Argania spinosa.

Saponins differing in structure from saponins from other plants are obtained from the residue accumulating in the extraction of oil from the seed kernels of Argania spinosa [Charrouf Z., et al.; Phytochemistry, 1992, 31; 2079-2086]. The saponins in question are known as arganin A, arganin B, arganin C, arganin D, arganin E, arganin F and misaponin A. The useful saponins arganin G, arganin H and arganin J can be isolated from the stem of the plant [Oulad-Ali A., et al.; J. Nat. Prod.; 1996, 59,193-195]. The aglycon of these saponins has the structure (I) shown below. The saponins mentioned differ in the sugar units at R1 and R3 and in a hydroxy group at R2. R3 is a tetrasaccharide while R1 is a mono- or disaccharide (for example 1,6-diglucose for arganin A and B).

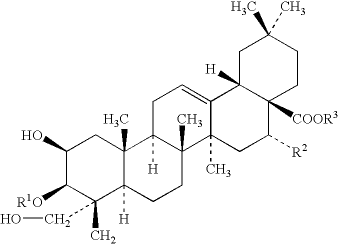

The saponins according to the invention show low toxicity in toxicological tests on mice and rats. In tests on human fibroblasts, the inventors were also able to demonstrate far lower toxicity by comparison with other saponins, for example from Gypsophila paniculata.

The saponins to be used in accordance with the invention correspond to arganin A, arganin B, arganin C, arganin D, arganin E, arganin F, misaponin A and to arganin G, arganin H and arganin J. They may be used as a mixture of two or more or in pure form in cosmetic and/or pharmaceutical preparations. Mixtures of arganin A, arganin B, arganin C, arganin D, arganin E, arganin F, misaponin A—the percentages of the saponins in the mixtures being variable—are particularly preferred. Extracts containing a high percentage of arganin A are preferably used. The use in accordance with the invention of extracts containing at least 6% by weight, preferably 8% by weight and more particularly at least 10% by weight arganin A, based on the dry weight of the extract, is distinguished by particularly pronounced effects.

Proteins

Proteins in the context of the invention are understood to be proteins which can be isolated from the plant Argania spinosa. It is preferred to extract the seed kernels, more particularly the defatted seed kernels after extraction of the oil. Accordingly, preparations containing native proteins obtained from an extract of the seed kernels, more particularly the defatted seed kernels of Argania spinosa, represent a particular embodiment of the invention.

In the context of the invention, the preferred extraction of the defatted seed kernels is understood to mean that the residue—a kind of cake—from the extraction of oil from the seed kernels of Argania spinosa is preferably extracted. This oil extraction residue which is preferably extracted contains 3 to 10% by weight of residual oil. The proteins according to the invention in this residue are completely separated from the residual oil. Besides proteins, other substances occurring naturally in Argania spinosa plants, which can be extracted under the same conditions, can be co-extracted.

In another embodiment of the invention, the preparations according to the invention contain native proteins which are obtained by extraction with water at a pH of or below 12, preferably between 3.5 and 6.5 and more particularly between 5.5 and 6.5 or between 3.5 and 5.5 and, optionally, subsequent drying, for example spray or freeze drying. The pH range selected is dependent upon the protein fraction to be isolated.

The native proteins which can be extracted from the plant Argania spinosa, more particularly from the seed kernels of the plant, may have molecular weights in the range from 10,000 Da to more than 500,000 Da. They may advantageously be divided into the following groups of molecular weight ranges. Native proteins with a molecular weight above 500,000 Da, native proteins with a molecular weight in the range from 170,000 to 250,000 Da and native proteins with a molecular weight in the range from 10,000 to 18,000 Da can be extracted.

Accordingly, other embodiments of the present invention are, on the one hand, preparations containing native proteins with a molecular weight above 500,000 Da, preparations containing native proteins with a molecular weight in the range from 170,000 Da to 250,000 Da and preferably in the range from 170,000 Da to 210,000 Da and preparations containing native proteins with a molecular weight in the range from 10,000 to 18,000 and preferably in the range from 13,000 to 16,000.

The percentage content of proteins in the extract used in accordance with the invention should preferably be at least 3% by weight, preferably at least 4% by weight and more particularly at least 5% by weight, based on the dry weight of the extract.

Extraction

The extracts to be used in accordance with the invention may be prepared by known methods of extracting plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Fresh or dried plants or parts thereof are suitable as the starting material although plants and/or plant parts which may be mechanically size-reduced before extraction are normally used. Any size reduction methods known to the expert, for example crushing with a mortar, may be used. In one particular embodiment, the extracts used are obtained by extraction of the stem, roots, leaves, flowers or fruits. Extraction of the seed kernels is particularly preferred.

Preferred solvents for the extraction process are organic solvents, water or mixtures of organic solvents and water, more particularly low molecular weight alcohols, esters, ethers, ketones or halogenated hydrocarbons with more or less large water contents (distilled or non-distilled), preferably aqueous alcoholic solutions with a temperature above 20° (hereinafter referred to as room temperature). Extraction with water, methanol, ethanol, acetone, propylene glycols, polyethylene glycols, ethyl acetate, dichloromethane, trichloromethane and mixtures thereof is particularly preferred. The extraction process is generally carried out at 20 to 100° C. and preferably at 80 to 85° C., more particularly at room temperature. In one possible embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried plants or dried plant parts (optionally defatted) are in the range from 3 to 20 and more particularly 4 to 16% by weight. The present invention includes the observation that the extraction conditions and the yields of the final extracts may be selected according to the desired application. If desired, the extracts may then be subjected, for example, to spray drying or freeze drying.

According to the invention, the extracts of this plant contain 10 to 99% by weight of saponins, preferably 15 to 70% by weight. The quantity in which the plant extracts are used in the anti-acne preparations, in the anti-seborrhoea preparations and in preparations with anti-5-α-reductase activity is determined by the concentration of the individual ingredients. The total quantity of plant extract present in the preparations is generally 0.01 to 25% by weight, preferably 0.03 to 5% by weight and more particularly 0.03 to 0.4% by weight, based on the final preparation.

The plant extracts used preferably contain proteins and saponins in the described quantity ranges in combination.

Commercial Applications

Cosmetic and/or pharmaceutical Preparations

The use of extracts from the plant Argania spinosa for the production of anti-acne preparations, anti-seborrhoea preparations and preparations with anti-5-α-reductase activity results in cosmetic and/or pharmaceutical preparations such as, for example, creams, gels, lotions, alcohol and water/alcohol solutions, emulsions, hair shampoos, hair lotions, foam baths, shower baths, wax/fat compounds, stick preparations, powders or ointments. These preparations may additionally contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, film formers, swelling agents, hydrotropes, solubilizers, preservatives, perfume oils, dyes and the like as further auxiliaries and additives.

The total percentage content of auxiliaries and additives may be from 1 to 70% by weight, preferably from 20 to 50% by weight and more particularly from 5 to 40% by weight, based on the final preparation of the cosmetic and/or pharmaceutical preparations. The preparations may be produced by standard hot or cold processes and are preferably produced by the phase inversion temperature method.

### EXAMPLES

### Example 1

### Preparation of the Saponin Crude Extract

0.3 kg of an Argania spinosa cake (from the seeds after extraction of the oil) were defatted with 1.98 g of hexane (1 hour at 80° C.). The defatted cake was then dried for 24 hours at room temperature. 0.12 kg of the defatted and dried cake were made up with 2 liters of 80% by vol. ethanol in a stirred vessel. The mixture was stirred for 16 hours at room temperature. The solids were then removed by filtration. The filtered solution forms the crude extract from which ethanol was removed by evaporation. Finally, the residue was freeze-dried.

Principle of the Test

Reconstructed epidermis—comparably to living skin—contains the entire enzymatic mechanism required for the metabolization of testosterone. The test with reconstructed epidermis in vitro is more appropriate because the 5-α-reductase remains in a biological system which comes very close to the in vivo system and which cannot be created by purified enzymes. This test is also relevant because the keratinocytes at the differentiation stage come closer to the in vivo test than when keratinocytes are used in monolayers [Bernard F.-X. et al., 2000, Int. J. Cosmetic Science, 22, 397-407, Expression of type 5-alpha-reductase and metabolism of testosterone in reconstructed human epidermis—SkinEthic: a new model for screening skin targeted androgen modulators].

Test setup:

Material:

Epidermis SkinEthic (17 days, 0.63 cm²) in culture, 37° C., 5% CO₂
Reference substance: Finasteride
Testosterone: [4-14-C] Testosterone (Amersham, CFA129, 56 mCi/mmole), 250 nCi/epidermis
Argania saponin extract of Example 1
   Treatment:

The epidermis was precultivated for 24 hours on plates with 24 positions. The experiments with the products and the reference substance Finasterid (10 μm) were carried out three times (3 epidermises per experiment). After treatment for 24 hours, the media of the subepidermis were renewed and replaced by 300 μl of fresh culture medium. 100 μl of radioactively marked testosterone solution were applied to the upper surface (horny layer) of the epidermis (TO). After another 24 hours, the subepidermis medium was removed for analysis.

The viability of the keratinocytes in the various epidermis samples was determined by the MTT method at the end of the test.

Extraction and analysis:

In order to determine the transepidermal diffusion, quantities of 20 μl of each culture medium were removed and counted in a liquid scintillation counter.

The steroids present in the culture medium were extracted for analysis of the metabolism and separated into their molecular derivatives by thin-layer chromatography (on silica gel). The quantity of transformed testosterone was determined by radioactive counting of the various spots using a phosphoimager.

Results:

The survivability of the treated epidermis and the transepidermal diffusion are shown in Table 1.

TABLE 1
Diffusion of ¹⁴C testosterone (and metabolites) by reconstructed
human epidermis (SkinEthic) and survivability of the tissue
at the end of the test (t = 24 hours)

| | | | Survival |
|---|---|---|---|
| Treatment | % ¹⁴C Testosterone | Nmole steroid | rate in % |
| Total testosterone | / | 4.5 | / |
| Control | 100 | 2.2 | 100 |
| Finasterid 10 μm | 110 | 2.4 | 101 |
| Argania extract of | | | |
| Example 1 | | | |
| 0.003% | 98 | 2.2 | 98 |
| 0.001% | 100 | 2.2 | 101 |

The survivability of untreated epidermis (control) and Finasterid-treated epidermis was identical with that of the extract-treated samples (2 concentrations).

For the transepidermal diffusion, approximately an average of the initial radioactivity was detected in the culture medium after incubation for 24 hours. The treatment with Finasterid resulted in a slight increase in the diffusion of steroids through the epidermis (110% of the control).

However, the analyzed extract did not influence the diffusion of steroids through the epidermis (value between 98 and 100% of the control).

The metabolization of testosterone is summarized in Table 2.

TABLE 2
Influence of the treatment with argania extract on
the production of DHT. Analysis by phosphoimager in
connection with the accumulated radioactivity

| Treatment | DHT formed in % |
|---|---|
| Control | 100 |
| Finasterid 10 μM | 8 |
| Argania saponin extract of Example 1 | |
| 0.001% | 61% |
| 0.003% | 74% |

The analyzed extract shows a distinct reduction in the production of DHT—39 and 26% inhibition. In a concentration of only 0.001 % by weight, there is already evidence of a significant inhibition of the 5-α-reductase activity without toxic effects influencing the survivability of the cells (determined by the MTT test).

1-11. (canceled)

12. A method for treating acne which comprises: applying to the affected area of the skin to be treated a composition containing an anti-acne effective amount of an extract of the plant Argania spinosa.

13. A method of treating seborrhoea which comprises: applying to the affected area of the skin to be treated a composition containing an anti-seborrhoea effective amount of an extract of the plant Argania spinosa.

14. A method for reducing the activity of anti-5-α-reductase which comprises: applying to the affected area of the skin to be treated a composition containing an anti-5-α-reductase effective amount of a preparation containing an anti-5-α-reductase effective amount of an extract of the plant Argania spinosa.

15. A method of claim 14 for treatment of unwanted hair growthin women which comprises: applying to the area of the skin where hair growth is not wanted a hair growth retarding effective amount of preparation containing an extract of the plant Argania spinosa.

16. The method of claim 12, wherein, the extract contains proteins and/or saponins.

17. The method of claim 12, wherein, the extract contains at least one saponin selected from the group consisting of arganin A, arganin B, arganin C, arganin D, arganin E, arganin F, arganin G, arganin H, arganin J and misaponin A.

18. The method of claim 12, wherein, the extract contains arganin A in a quantity of at least 6% by weight, based on the dry weight of the extract, as the saponin.

19. The method of claim 12, wherein, the extract contains at least 3% by weight of proteins, based on the dry weight of the extract.

20. The method of claim 12, wherein, the effective amount of the extract is from 0.01 to 25% by weight, expressed as dry weight, based on the weight of the preparation.

21. The method of claim 12, wherein, the extract is obtained by extraction of at least one plant part selected from the group consisting of leaves, stems, bark, flowers, fruits, fruit flesh and seed kernels.

22. The method of claim 12, wherein, the extract is obtained by extraction of at least one member selected from the group consisting of seed kernels and defatted seed kernels from the fruit of the plant Argania spinosa.

23. The method of claim 13, wherein, the extract contains proteins and/or saponins.

24. The method of claim 14, wherein, the extract contains proteins and/or saponins.

25. The method of claim 15, wherein, the extract contains proteins and/or saponins.

26. The method of claim 13, wherein, the extract contains at least one saponin selected from the group consisting of arganin A, arganin B, arganin C, arganin D, arganin E, arganin F, arganin G, arganin H, arganin J and misaponin A.

27. The method of claim 14, wherein, the extract contains at least one saponin selected from the group consisting of arganin A, arganin B, arganin C, arganin D, arganin E, arganin F, arganin G, arganin H, arganin J and misaponin A.

28. The method of claim 15, wherein, the extract contains at least one saponin selected from the group consisting of arganin A, arganin B, arganin C, arganin D, arganin E, arganin F, arganin G, arganin H, arganin J and misaponin A.

29. The method of claim 13, wherein, the effective amount of the extract is from 0.01 to 25% by weight, expressed as dry weight, based on the weight of the preparation.

30. The method of claim 14, wherein, the effective amount of the extract is from 0.01 to 25% by weight, expressed as dry weight, based on the weight of the preparation.

31. The method of claim 15, wherein, the effective amount of the extract is from 0.01 to 25% by weight, expressed as dry weight, based on the weight of the preparation.

32. A cosmetic or therapeutic composition comprising a dermatologically acceptable carrier and an extract of Argania spinosa whereby application to the skin treats at least one condition selected from the group consisting of acne, seborrhoea, and unwanted hair growth.
